(19) United States
(12) Patent Application Publication
(10) Pub. No.: US 2026/0038645 A1
(43) Pub. Date: Feb. 5, 2026

(54) METHOD AND APPARATUS FOR CONSTRUCTING CATALYST SYSTEM RELAXED ENERGY PREDICTION MODEL

(71) Applicant: Tencent Technology (Shenzhen) Company Limited, Shenzhen (CN)

(72) Inventors: Geyan YE, Shenzhen (CN); Wei LIU, Shenzhen (CN)

(21) Appl. No.: 19/356,897

(22) Filed: Oct. 13, 2025

Related U.S. Application Data
(63) Continuation of application No. PCT/CN2024/116572, filed on Sep. 3, 2024.

(30) Foreign Application Priority Data
Sep. 14, 2023 (CN) 202311190183.3
Sep. 14, 2023 (CN) 202311190486.5

(51) Int. Cl. G16C 20/30; G16C 20/70

(52) U.S. Cl. CPC G16C 20/30; G16C 20/70

(57) ABSTRACT

A method for constructing a catalyst system relaxed energy prediction model is performed by a computing device, and the method includes: obtaining a first training data set including a plurality of pieces of first training data, each piece including a first training sample and a corresponding first sample label; training a catalyst system energy prediction model by using the first training data set, to obtain a pre-trained catalyst system energy prediction model; constructing a catalyst system relaxed energy initial prediction model based on the pre-trained catalyst system energy prediction model; obtaining a second training data set including a plurality of pieces of second training data, each piece including a second training sample and a corresponding second sample label; and training the catalyst system relaxed energy initial prediction model by using the second training data set, to obtain a catalyst system relaxed energy prediction model.

### CROSS-REFERENCE TO RELATED APPLICATIONS

This application is a continuation application of PCT Patent Application No. PCT/CN2024/116572, entitled “METHOD AND APPARATUS FOR CONSTRUCTING CATALYST SYSTEM RELAXED ENERGY PREDICTION MODEL” filed on Sep. 3, 2024, which claims priorities to (i) Chinese Patent Application No. 2023111904865, entitled “METHOD AND APPARATUS FOR CONSTRUCTING CATALYST SYSTEM RELAXED ENERGY PREDICTION MODEL” filed on Sep. 14, 2023 and (ii) Chinese Patent Application No. 2023111901833, entitled “METHOD AND APPARATUS FOR CONSTRUCTING CATALYST SYSTEM RELAXED ENERGY PREDICTION MODEL” filed on Sep. 14, 2023, all of which are incorporated herein by reference in their entireties.

### FIELD OF THE TECHNOLOGY

This application relates to the field of computer technologies, and in particular, to a method and an apparatus for constructing a catalyst system relaxed energy prediction model, a computing device, a non-transitory computer-readable storage medium, and a computer program product.

### BACKGROUND OF THE DISCLOSURE

As an important auxiliary means of chemical reactions, catalysts are widely used in fields such as a chemical industry and a manufacturing industry. Selection of an appropriate catalyst for a chemical reaction is an important means of controlling a speed of the chemical reaction and ensuring safety of the reaction. In a conventional method, the appropriate catalyst is usually selected from a huge quantity of candidate materials through a large quantity of experiments. The method is time-consuming and labor-consuming, and also causes a great waste. Therefore, the experimental personnel hope to predict some key indicators of the catalyst in advance, to reduce the quantity of candidate catalysts in mass-selection, to quickly find the appropriate catalyst. Relaxed energy may indicate energy released by an adsorbate-catalyst system from an initial state to a steady state, and is a key indicator for measuring performance of a catalyst. However, a conventional relaxed energy prediction method based on quantum mechanics has high calculation complexity and a huge calculation amount, severely affecting research and development efficiency of the catalyst. In recent years, with the development of computer technologies, using a deep learning method to construct a relaxed energy prediction model to predict relaxed energy of the adsorbate-catalyst system attracts increasing attention.

### SUMMARY

This application provides a method and an apparatus for constructing a catalyst system relaxed energy prediction model, a computing device, a non-transitory computer-readable storage medium, and a computer program product.

According to an aspect of this application, a method for constructing a catalyst system relaxed energy prediction model is provided, and is performed by a computing device. The method includes:
obtaining a first training data set, the first training data set including a plurality of pieces of first training data, each piece of first training data including a first training sample and a corresponding first sample label;
training a catalyst system energy prediction model by using the first training data set, to obtain a pre-trained catalyst system energy prediction model;
constructing a catalyst system relaxed energy initial prediction model based on the pre-trained catalyst system energy prediction model;
obtaining a second training data set, the second training data set including a plurality of pieces of second training data, each piece of second training data including a second training sample and a corresponding second sample label; and
training the catalyst system relaxed energy initial prediction model by using the second training data set, to obtain a catalyst system relaxed energy prediction model.

According to another aspect of this application, a method for predicting relaxed energy of a catalyst system is provided, including: obtaining system structure information of a target catalyst system; and inputting the system structure information of the target catalyst system to the catalyst system relaxed energy prediction model in any one of the foregoing embodiments, to obtain relaxed energy information of the target catalyst system.

According to another aspect of this application, a computing device is provided, including: a memory, configured to store computer-executable instructions; and a processor, configured to perform, when the computer-executable instructions are executed by the processor, operations of the method for constructing a catalyst system relaxed energy prediction model according to some embodiments of this application.

According to another aspect of this application, a non-transitory computer-readable storage medium is provided, having computer-executable instructions stored therein. The computer-executable instructions, when executed, implement operations of the method for constructing a catalyst system relaxed energy prediction model according to some embodiments of this application.

These and other advantages of this application become clear according to the embodiments described below, and these and other advantages of this application are explained with reference to the embodiments described below.

### BRIEF DESCRIPTION OF THE DRAWINGS

To describe the technical solutions in embodiments of this application or in the conventional technology more clearly, the following briefly describes the accompanying drawings required for describing the embodiments or the conventional technology. Apparently, the accompanying drawings in the following description show only the embodiments of this application, and a person of ordinary skill in the art may derive other drawings from the disclosed accompanying drawings without creative efforts.

FIG. 1 is a schematic diagram of a research and development procedure of a catalyst according to an embodiment of this application.

FIG. 2 shows an exemplary application scenario of a method for constructing a catalyst system relaxed energy prediction model according to an embodiment of this application.

FIG. 3 is a schematic flowchart of a method for constructing a catalyst system relaxed energy prediction model according to an embodiment of this application.

FIG. 4 is a schematic flowchart of training a catalyst system energy prediction model by using a first training data set, to obtain a pre-trained catalyst system energy prediction model according to an embodiment of this application.

FIG. 5 is a schematic diagram of training a catalyst system energy prediction model according to an embodiment of this application.

FIG. 6 is a schematic flowchart of training a catalyst system relaxed energy initial prediction model by using a second training data set, to obtain a catalyst system relaxed energy prediction model.

FIG. 7 is a schematic diagram of training a catalyst system relaxed energy prediction model according to an embodiment of this application.

FIG. 8 is a schematic diagram of training a multi-task catalyst system relaxed energy prediction model according to an embodiment of this application.

FIG. 9 is a schematic diagram of training a catalyst system relaxed energy prediction model according to an embodiment of this application.

FIG. 10 is a schematic diagram of a method for constructing a catalyst system relaxed energy prediction model according to an embodiment of this application, where the catalyst system relaxed energy prediction model is constructed through pre-training without multi-task training.

FIG. 11 is a schematic diagram of a method for constructing a multi-task catalyst system relaxed energy prediction model according to an embodiment of this application.

FIG. 12 is a diagram of comparison between effects of a catalyst system relaxed energy prediction model and a control group according to an embodiment of this application.

FIG. 13 is an exemplary structural block diagram of an apparatus for constructing a catalyst system relaxed energy prediction model according to an embodiment of this application.

FIG. 14 is a schematic structural diagram of an apparatus for predicting relaxed energy of a catalyst system according to an embodiment of this application.

FIG. 15 shows an exemplary system including an exemplary computing device representing one or more systems and/or devices that can implement various methods described in this specification.

### DESCRIPTION OF EMBODIMENTS

Technical solutions in embodiments of this application are clearly and completely described below with reference to the accompanying drawings in the embodiments of this application. Apparently, the described embodiments are merely some rather than all of the embodiments of this application. All other embodiments obtained by a person skilled in the art based on the embodiments of this application without creative efforts shall fall within the protection scope of this application. A same reference numeral in the accompanying drawings represents same or similar components, and therefore repeated descriptions of the components are appropriately omitted.

In addition, the described features, structures, or characteristics may be combined in one or more embodiments in any appropriate manner. In the following descriptions, many specific details are provided to provide a full understanding of the embodiments of this application. However, a person skilled in the art will be aware that the technical solutions of this application may be implemented without one or more of the specific details, or other methods, components, apparatuses, steps, or the like may be used. In other cases, well-known methods, apparatuses, implementations, or operations are not shown or described in detail to avoid obscuring aspects of this application.

Block diagrams shown in the accompanying drawings are merely functional entities and do not necessarily correspond to physically separate entities. To be specific, the functional entities may be implemented in a software form, or in one or more hardware modules or integrated circuits, or in different networks and/or processor apparatuses and/or microcontroller apparatuses.

Although the terms “first”, “second”, and “third” may be used in this specification to describe various components, the components are not limited by the terms. The terms are used to distinguish one component from another component. Therefore, a first component discussed below may be referred to as a second component without departing from the teaching of the concept of this application. As used in this specification, the term “and/or” and similar terms include all combinations of any one, multiple, and all of associated listed items.

Before the embodiments of this application are described in detail, some terms in embodiments of this application are first described below, to facilitate understanding of a person skilled in the art.

A catalyst is generally a material that improves a reaction rate without changing a total standard Gibbs free energy change of a reaction. Generally, the catalyst is a material that participates in a middle process of a chemical reaction and that can also selectively change a rate of the chemical reaction, but a quantity and a chemical property of the material basically remain unchanged before and after the reaction. Alternatively, the catalyst may be expressed as a material that can improve a rate of a chemical reaction during the chemical reaction without changing chemical equilibrium, and a quality and a chemical property of the material do not change before and after the chemical reaction. According to statistics, catalysts are used in more than 90% of industrial processes, such as chemical, petrochemical, biochemical, and environmental protection industrial processes. There are many types of catalysts, which may be classified into a liquid catalyst and a solid catalyst based on states; and may be classified into a homogeneous catalyst and a heterogeneous catalyst based on phase states of a reaction system. The homogeneous catalyst includes acid, alkali, soluble transition metal compound, and peroxide catalysts. The catalysts play an extremely important role in the modern chemical industry. For example, an iron catalyst is used in production of synthetic ammonia, a vanadium catalyst is used in production of sulfuric acid, and different catalysts are used in production of three major synthetic materials, such as polymerization of ethylene and preparation of rubber from butadiene. Searching for an appropriate catalyst for a chemical reaction is still an important topic in the field of the chemical industry, and is also an important means of controlling a reaction rate and improving safety of the reaction.

A catalyst system, also referred to as an adsorbate-catalyst system, is a system including a plurality of atoms or molecules as a catalyst, and may also include atoms or molecules that are adsorbed on the catalyst and that are used as a reactant. The catalyst system includes the plurality of atoms or molecules. In addition, with continuous reaction of the reactant as an adsorbate, a force between the atoms or molecules also changes continuously, and energy of the system also changes accordingly, until the reaction is completed and the system tends to be in a steady state. Therefore, each of microstructures of the catalyst system corresponds to one piece of system energy, and in this case, a corresponding force relationship exists between atoms or molecules in the microstructure. Usually, for a catalyst system, a difference between system energy corresponding to an initial structure and system energy corresponding to a final structure after reaction completion is used as relaxed energy of the system, for measuring performance of a catalyst. Greater relaxed energy of the catalyst system indicates better performance of the catalyst.

A graph neural network (GNN) is a collective name of algorithms that learn, by using a neural network, graph structure data, extract and mine features and modes in the graph structure data, and meet requirements on graph learning tasks such as clustering, classification, prediction, segmentation, and generation. Policies are formulated on nodes and edges in a graph, so that the GNN converts the graph structure data into a standard representation, and inputs the standard representation into a plurality of different neural networks for training, to achieve good effects on tasks such as node classification, edge information propagation, and graph clustering.

A topology diagram is a simple and regular representation of associations between entities, and displays quantized information accordingly. The topology diagram transmits the quantized information in a form of a diagram, and is an effective representation form for representing the associations between the entities.

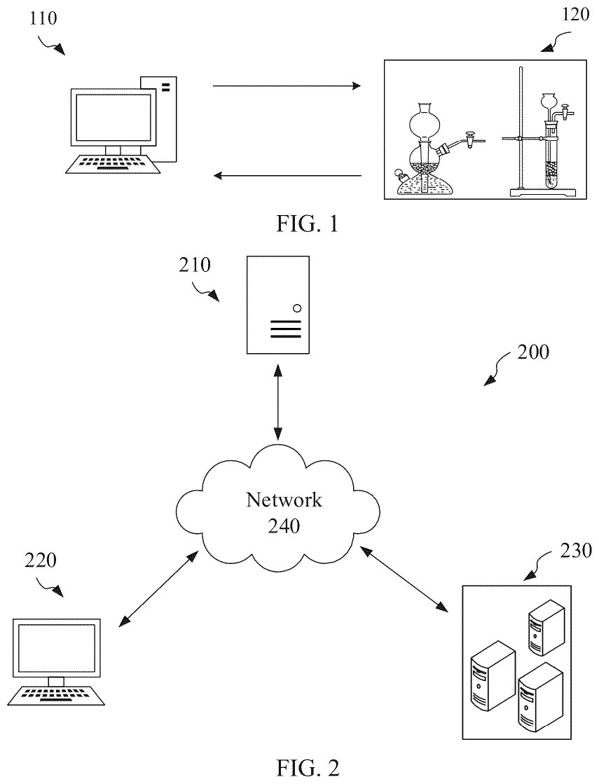

FIG. 1 is a schematic diagram of a research and development procedure of a catalyst according to some embodiments of this application. As shown in FIG. 1, during research and development of a catalyst, a computing device 110 and an experimental device 120 are usually used to perform efficient filtering on catalysts. First, the computing device is used to predict relaxed energy of a large quantity of catalysts, to select catalysts having a better prediction result. Then, the experimental device 120 is used to perform experimental verification on the selected catalysts, to sift out a catalyst whose experimental performance meets expectation. According to such a catalyst filtering method by using the computing device, research and development efficiency of the catalyst can be greatly improved, but high requirements on precision and a speed at which the computing device 110 predicts the relaxed energy are raised. Therefore, a catalyst system relaxed energy prediction model needs to be constructed. However, due to a complex atomic structure of the catalyst system, a rapid structural change during a chemical reaction, and the like, it is difficult to construct the catalyst system relaxed energy prediction model, and the precision is poor. In a related technical solution, a model may be constructed by using a quantum mechanical method, but the method has many shortcomings. A quantum mechanical model usually implements molecular simulation by using a density functional theory method, and then predicts relaxed energy of a catalyst system based on the molecular simulation. Construction and running of such a model require an operation on each molecule or atom. Therefore, a calculation amount is very large and a corresponding speed is low for both modeling and running of the model. Consequently, it is difficult to meet an actual application requirement.

Therefore, this application provides a method for constructing a catalyst system relaxed energy prediction model, to construct a catalyst system relaxed energy prediction model. The model may be run on the computing device 110, to precisely predict relaxed energy of a catalyst system.

FIG. 2 shows an exemplary application scenario 200 of a method for constructing a catalyst system relaxed energy prediction model according to some embodiments of this application. The application scenario 200 may include a server 210, a terminal device 220, and a server 230. The server 210, the terminal device 220, and the server 230 are communicatively coupled through a network 240. The network 240 may be, for example, a wide area network (WAN), a local area network (LAN), a wireless network, a public telephone network, an intranet, or any other type of network well-known by a person skilled in the art.

In some embodiments, the method for constructing a catalyst system relaxed energy prediction model may be mainly run on the server 210. A first training data set is obtained from the server 210, the first training data set including a plurality of pieces of first training data, each piece of first training data including a first training sample and a corresponding first sample label, the first training sample including first catalyst system structure information, and the first sample label including system energy information corresponding to the first catalyst system structure information. In some embodiments, the first training data set may be stored on the server 210, or may be obtained from another server or terminal through the network 240. Then, a catalyst system energy prediction model is trained on the server 210 by using the first training data set, to obtain a pre-trained catalyst system energy prediction model, and a catalyst system relaxed energy initial prediction model is constructed based on the pre-trained catalyst system energy prediction model. Then, a second training data set is obtained from the server 210, the second training data set including a plurality of pieces of second training data, each piece of second training data including a second training sample and a corresponding second sample label, the second training sample including second catalyst system structure information, and the second sample label including relaxed energy information corresponding to the second catalyst system structure information. In some embodiments, the second training data set may be stored on the server 210, or may be obtained from another server or terminal through the network 240. Finally, the catalyst system relaxed energy initial prediction model is trained on the server 210 by using the second training data set, to obtain a catalyst system relaxed energy prediction model.

In some embodiments, the method for constructing a catalyst system relaxed energy prediction model may alternatively be mainly run on the terminal device 220 or the server 230. The server 210, the terminal device 220, and the server 230 each may include a medium and/or a device that can persistently store information, and/or a tangible storage apparatus. Therefore, a non-transitory computer-readable storage medium is a non-signal-carrying medium. The computer-readable storage medium includes hardware such as volatile and non-volatile media, removable and non-removable media, and/or a storage device implemented by using a method or technology suitable for storing information (such as computer-readable instructions, a data structure, a program module, a logical element/circuit, or other data). A person of ordinary skill in the art understands that an instance of the server 210 may be an independent physical server, may be a server cluster including a plurality of physical servers or a distributed system, or may be a cloud server providing basic cloud computing services, such as a cloud service, a cloud database, cloud computing, a cloud function, cloud storage, a network service, cloud communication, a middleware service, a domain name service, a security service, a content delivery network (CDN), big data, and an artificial intelligence platform. The terminal and the server may be directly or indirectly connected in a wired or wireless communication manner. This is not limited herein in this application. The server 210 may present a to-be-determined data allocation policy to a developer through the terminal device 220, and perform interaction with the developer, to visually determine a development policy.

The terminal device 220 may be any type of mobile computing device, including a mobile computer (for example, a personal digital assistant (PDA), a laptop computer, a notebook computer, a tablet computer, or a netbook), a mobile phone (for example, a cellular phone or a smartphone), a wearable computing device (for example, a smart watch or a head-mounted device, including smart glasses), or another type of mobile device. In some embodiments, the terminal device 220 and the server 230 may alternatively be fixed computing devices, such as desktop computers, game consoles, and smart televisions. In addition, when the application scenario 200 includes a plurality of terminal devices 220, the plurality of terminal devices 220 may be computing devices of a same type or different types.

As shown in FIG. 2, the terminal device 220 may include a display screen and a terminal application that may perform interaction with a terminal user via the display screen. The terminal application may be a local application program, a Web application program, or an applet (LiteApp, for example, a mobile phone applet or a WeChat applet) used as a lightweight application. When the terminal application is a local application program that needs to be installed, the terminal application may be installed in the terminal device 220. When the terminal application is a Web application program, the terminal application may be accessed by using a browser. When the terminal application is an applet, the terminal application may be directly opened on the terminal device 220 in a manner like searching for information related to the terminal application (such as a name of the terminal application) or scanning a graphic code (for example, a bar code or a two-dimensional code) of the terminal application, without installing the terminal application.

In some embodiments, the application scenario 200 may be a distributed system including the server 230, and the distributed system may form, for example, a blockchain system. A blockchain is a new application mode of computer technologies such as distributed data storage, peer-to-peer transmission, a consensus mechanism, and an encryption algorithm. The blockchain is essentially a decentralized database and is a string of data blocks generated through association by using a cryptographic method. Each data block includes information about a batch of network transactions, to perform verification on validity of the information of the data block (anti-counterfeiting) and generate a next data block. The blockchain may include an underlying blockchain platform, a platform product service layer, and an application service layer.

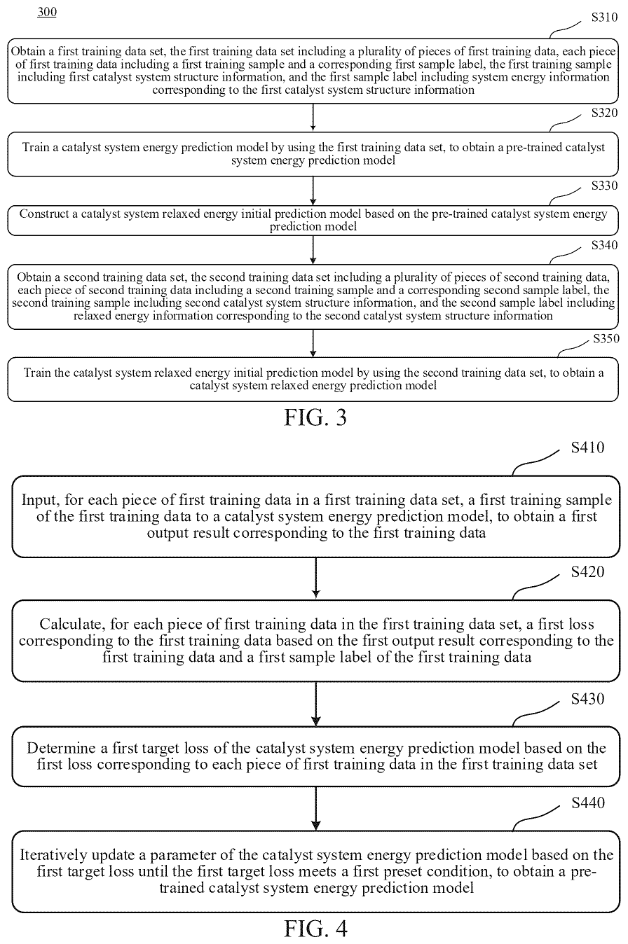

FIG. 3 is a schematic flowchart of a method 300 for constructing a catalyst system relaxed energy prediction model according to some embodiments of this application. As shown in FIG. 3, the method 300 includes operation S310, operation S320, operation S330, operation S340, and operation S350.

Operation S310: Obtain a first training data set, the first training data set including a plurality of pieces of first training data, each piece of first training data including a first training sample and a corresponding first sample label, the first training sample including first catalyst system structure information, and the first sample label including system energy information corresponding to the first catalyst system structure information.

The first catalyst system structure information may be structure information of a catalyst system represented by the first training sample, and may represent a structure of the catalyst system. The system energy information corresponding to the first catalyst system structure information may represent system energy of the catalyst system represented by the first catalyst system structure information.

In some embodiments, the system energy information corresponding to the first catalyst system structure information may be obtained through measurement or quantum mechanical calculation and then stored, to be invoked during use. In other embodiments, the first sample label may further include atomic force information corresponding to the first catalyst system structure information. The atomic force information may be obtained through measurement or quantum mechanical calculation and then stored, to be invoked during use.

Operation S320: Train a catalyst system energy prediction model by using the first training data set, to obtain a pre-trained catalyst system energy prediction model.

In some embodiments, the catalyst system energy prediction model may include a graph neural network. Training the catalyst system energy prediction model by using the first training data set includes training a parameter of the graph neural network by using the first training data set, to obtain the graph neural network that can predict the energy of the catalyst system based on the structure of the catalyst system.

Operation S330: Construct a catalyst system relaxed energy initial prediction model based on the pre-trained catalyst system energy prediction model.

In some embodiments, the pre-trained catalyst system energy prediction model may be used as the catalyst system relaxed energy initial prediction model. Because the pre-trained catalyst system energy prediction model is a multi-task model (where the model predicts both the system energy and a system force of the catalyst system based on the structure of the catalyst system), a parameter setting process for the model is stricter than that for training of a single-task model (for example, can be more comprehensive during loss calculation). In this way, parameter setting efficiency of the model is higher, convergence is faster, and an obtained parameter is also more adaptive.

An output of the pre-trained catalyst system energy prediction model includes an output similar to the output of the catalyst system relaxed energy initial prediction model (for example, includes a one-dimensional output and a matrix output). Therefore, the pre-trained catalyst system energy prediction model is used as the catalyst system relaxed energy initial prediction model, to determine a good initial parameter value for subsequent model construction.

Operation S340: Obtain a second training data set, the second training data set including a plurality of pieces of second training data, each piece of second training data including a second training sample and a corresponding second sample label, the second training sample including second catalyst system structure information, and the second sample label including relaxed energy information corresponding to the second catalyst system structure information.

In some embodiments, the second catalyst system structure information may be structure information of the catalyst system represented by the second training sample, and may represent a structure of the catalyst system. The relaxed energy information corresponding to the second catalyst system structure information may represent relaxed energy of the catalyst system represented by the second catalyst system structure information.

In some embodiments, the relaxed energy information corresponding to the second catalyst system structure information may be obtained through measurement or quantum mechanical calculation and then stored, to be invoked during use. The second sample label may further include atomic displacement information corresponding to the second catalyst system structure information. The atomic displacement information corresponding to the second catalyst system structure information may be obtained through measurement or quantum mechanical calculation and then stored, to be invoked during use.

Operation S350: Train the catalyst system relaxed energy initial prediction model by using the second training data set, to obtain a catalyst system relaxed energy prediction model.

In some embodiments, the catalyst system relaxed energy initial prediction model may include the graph neural network. Training the catalyst system relaxed energy initial prediction model by using the second training data set includes further training the pre-trained parameter of the graph neural network by using the second training data set, to obtain the graph neural network that can predict the relaxed energy of the catalyst system based on the structure of the catalyst system.

Therefore, in the method 300, a catalyst system relaxed energy initial prediction model is constructed based on a pre-trained catalyst system energy prediction model, so that the catalyst system relaxed energy initial prediction model can be quickly determined. Then, the catalyst system relaxed energy initial prediction model is adjusted by using a second training data set, to obtain a catalyst system relaxed energy prediction model, so that the catalyst system relaxed energy prediction model with high precision and a fast response can be quickly obtained. It can be learned that, because the catalyst system relaxed energy prediction model constructed by using the method 300 uses a two-stage training process, precision of relaxed energy prediction is significantly improved, thereby avoiding a problem of low calculation precision in the related technology. In addition, the catalyst system relaxed energy prediction model constructed according to some embodiments of this application may directly predict relaxed energy based on initial structure information of a system without iterative calculation, and has a fast calculation speed, thereby avoiding problems such as high calculation complexity, complex calculation processes, and slow calculation speeds in a conventional quantum mechanical method and the related technology. Therefore, according to the method 300, a high-quality catalyst system relaxed energy prediction model can be constructed quickly and easily.

In some embodiments, the first sample label may further include the atomic force information corresponding to the first catalyst system structure information, and the second sample label may further include the atomic displacement information corresponding to the second catalyst system structure information.

The atomic force information may represent information about an atomic force in the catalyst system having the system structure represented by the first catalyst system structure information, and may include a force magnitude and a force direction. The atomic displacement information represents information about atomic displacement in the catalyst system. Atomic displacement of an atom in the catalyst system may be obtained by subtracting an initial position of the atom from a final position of the atom (that is, a position of the atom when the system is in a relaxed state). The atomic displacement information may include an atomic displacement magnitude and a displacement direction.

In some embodiments, because the second training data includes a plurality of pieces of expected prediction data (for example, relaxed energy and atomic displacement), the model trained by using the second data set can simultaneously process a plurality of tasks. In addition, because the catalyst system relaxed energy initial prediction model is a multi-task model, by adjusting a parameter of the model by using the second training data, fast convergence can be achieved, and prediction precision of an obtained model is high.

In the embodiments, a multi-task catalyst system relaxed energy initial prediction model is constructed based on a pre-trained multi-task catalyst system energy prediction model, so that the multi-task catalyst system relaxed energy initial prediction model can be quickly determined. Then, the multi-task catalyst system relaxed energy initial prediction model is adjusted by using a second training data set, to obtain a multi-task catalyst system relaxed energy prediction model. Both training at a first stage and training at a second stage are training models to adapt to multiple tasks (for example, the first stage includes two tasks: predicting system energy and predicting an atomic force, and the second stage includes two tasks: predicting relaxed energy and predicting atomic displacement), and dimensions of expected outputs of the models at the first stage and the second stage correspond to each other (for example, data dimensions of the system energy and the relaxed energy are the same, and data dimensions of the atomic force and the atomic displacement are the same). Therefore, a training process at the second stage can be quicker and more efficient, and prediction precision of the final catalyst system relaxed energy prediction model is also higher. It can be learned that, because the catalyst system relaxed energy prediction model uses a two-stage training process, precision of relaxed energy prediction is significantly improved, thereby avoiding a problem of low calculation precision in the related technology. In addition, the catalyst system relaxed energy prediction model may directly predict relaxed energy based on initial structure information of a system without iterative calculation, and has a fast calculation speed, thereby avoiding problems such as high calculation complexity, complex calculation processes, and slow calculation speeds in a conventional quantum mechanical method and the related technology. In addition, because training is performed for multiple tasks at both the first stage and the second stage, efficiency of training the catalyst system relaxed energy prediction model is higher, and prediction precision of the obtained model is better.

FIG. 4 is a schematic flowchart of training a catalyst system energy prediction model by using a first training data set, to obtain a pre-trained catalyst system energy prediction model. As shown in FIG. 4, in some embodiments, operation S320 includes operation S410, operation S420, operation S430, and operation S440.

Operation S410: Input, for each piece of first training data in the first training data set, the first training sample of the first training data to the catalyst system energy prediction model, to obtain a first output result corresponding to the first training data.

Operation S420: Calculate, for each piece of first training data in the first training data set, a first loss corresponding to the first training data based on the first output result corresponding to the first training data and the first sample label of the first training data. In some embodiments, operation S420 includes: calculating, for each piece of first training data in the first training data set, a first sub-loss corresponding to the first training data based on a catalyst system energy prediction result corresponding to the first training data and the system energy information of the first training data.

Operation S430: Determine a first target loss of the catalyst system energy prediction model based on the first loss corresponding to each piece of first training data in the first training data set. In some embodiments, the first target loss may be calculated by using a loss function.

In some embodiments, when the first sample label further includes the atomic force information corresponding to the first catalyst system structure information, the first output result includes the catalyst system energy prediction result and an atomic force information prediction result; and operation S430 includes: calculating, for each piece of first training data in the first training data set, a second loss corresponding to the first training data based on the atomic force information prediction result corresponding to the first training data and the atomic force information of the first training data; and determining the first target loss of the catalyst system energy prediction model based on the first loss and the second loss corresponding to each piece of first training data in the first training data set. When the first target loss is calculated, all first losses and all second losses obtained through calculation may be summed up, and a summation result is used as the first target loss.

Operation S440: Iteratively update a parameter of the catalyst system energy prediction model based on the first target loss until the first target loss meets a first preset condition, to obtain the pre-trained catalyst system energy prediction model. Therefore, the pre-trained catalyst system energy prediction model may precisely predict the energy of the catalyst system based on the structure of the catalyst system.

In some embodiments, the catalyst system energy prediction model includes a catalyst system energy-force prediction model, and the first sample label further includes the atomic force information corresponding to the first catalyst system structure information. In some embodiments, the atomic force information corresponding to the first catalyst system structure information may be obtained through experimental measurement or quantum mechanical calculation and then stored, to be invoked when needed. The first output result includes the catalyst system energy prediction result and the atomic force information prediction result. In some embodiments, the catalyst system energy prediction result may be represented by a scalar, and the atomic force information prediction result may be represented by a matrix.

In some embodiments, the first preset condition includes at least one of the following: A current first target loss is less than a predetermined threshold; and a quantity of iterations corresponding to the current first target loss reaches a predetermined quantity. For example, the predetermined quantity is set to 500. When the quantity of iterations exceeds 500, the iteration is stopped.

In some embodiments, operation S420 includes the following operations:

First, for each piece of first training data in the first training data set, the first sub-loss is calculated based on the catalyst system energy prediction result corresponding to the first training data and the system energy information of the first training data. In some embodiments, the first sub-loss may be obtained by calculating a difference between the catalyst system energy prediction result corresponding to the first training data and the system energy information of the first training data.

Then, for each piece of first training data in the first training data set, a second sub-loss corresponding to the first training data is calculated based on the atomic force information prediction result corresponding to the first training data and the atomic force information of the first training data. In some embodiments, the second sub-loss may be obtained by calculating a difference in each of dimensions between the matrix that represents the atomic force information prediction result and a matrix that represents the atomic force information and then combining differences into a scalar.

Finally, for each piece of first training data in the first training data set, the first loss is determined based on the first sub-loss and the second sub-loss corresponding to the first training data. In some embodiments, determining the first loss based on the first sub-loss and the second sub-loss may include calculating a sum of the first sub-loss and the second sub-loss, to obtain the first loss.

In some embodiments, atomic force information of the catalyst system may be represented in a form of a three-dimensional matrix, and three-dimensional data separately represents forces of an atom in three directions, that is, a direction x, a direction y, and a direction z. In addition, system structure information of the catalyst system may also be represented in a form of a three-dimensional matrix, and three-dimensional data separately represents three-dimensional coordinates of an atom. It can be learned that, input and output dimensions are very close to each other, facilitating training of a parameter of the catalyst system energy-force prediction model.

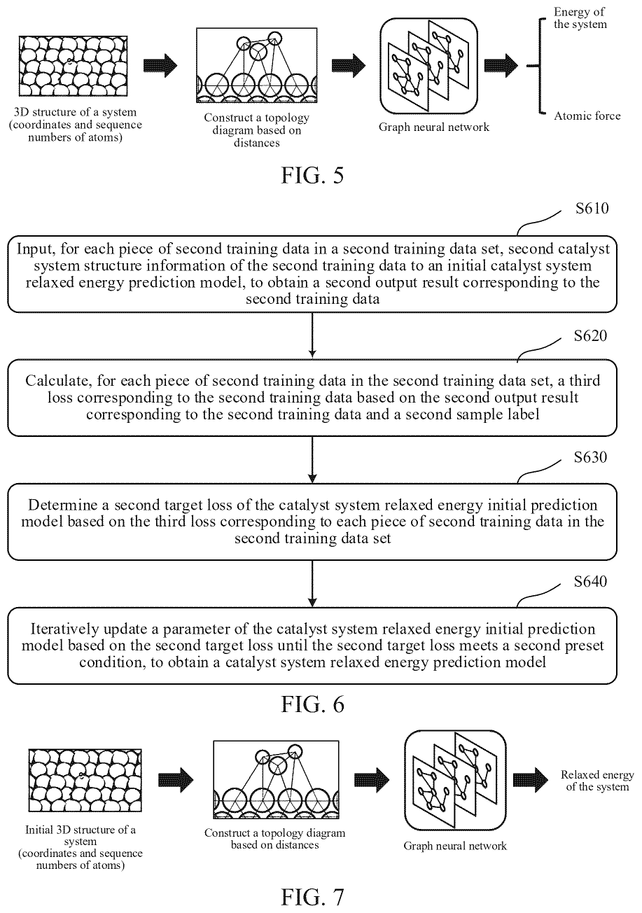

FIG. 5 is a schematic diagram of training a catalyst system energy prediction model according to some embodiments of this application. As shown in FIG. 5, first, a 3D structure (including coordinates and sequence numbers of atoms) of a catalyst system, and system energy and an atomic force corresponding to the 3D structure of the catalyst system are obtained. For example, the system energy and the atomic force corresponding to the 3D structure of the catalyst system may be calculated through quantum mechanics. Then, a topology diagram of the catalyst system is constructed based on distances between the atoms. Then, a parameter of a graph neural network is trained by using the topology diagram of the structure of the catalyst system as an input and using the system energy and the atomic force corresponding to the structure of the catalyst system as an expected output, to obtain the catalyst system energy prediction model.

FIG. 6 is a schematic flowchart of training a catalyst system relaxed energy initial prediction model by using a second training data set, to obtain a catalyst system relaxed energy prediction model. As shown in FIG. 6, in some embodiments, operation S350 includes operation S610, operation S620, operation S630, and operation S640.

Operation S610: Input, for each piece of second training data in the second training data set, the second catalyst system structure information of the second training data to the catalyst system relaxed energy initial prediction model, to obtain a second output result corresponding to the second training data.

In some embodiments, the second output result includes a catalyst system relaxed energy prediction result and an atomic displacement information prediction result. In some embodiments, the catalyst system relaxed energy prediction result is represented by a scalar, and the atomic displacement information prediction result is represented by a matrix.

Operation S620: Calculate, for each piece of second training data in the second training data set, a third loss corresponding to the second training data based on the second output result corresponding to the second training data and the second sample label.

In some embodiments, the second output result includes the catalyst system relaxed energy prediction result; and operation S620 includes: calculating, for each piece of second training data in the second training data set, the third loss corresponding to the second training data based on the catalyst system relaxed energy prediction result corresponding to the second training data and the relaxed energy information of the second training data.

In some embodiments, the third loss may be obtained by calculating a difference between the catalyst system relaxed energy prediction result and the relaxed energy information of the second training data.

Operation S630: Determine a second target loss of the catalyst system relaxed energy initial prediction model based on the third loss corresponding to each piece of second training data in the second training data set. In some embodiments, the second target loss may be obtained by using a loss function.

In some embodiments, the second sample label further includes the atomic displacement information corresponding to the second catalyst system structure information; the second output result further includes the atomic displacement information prediction result; and operation S630 includes: calculating, for each piece of second training data in the second training data set, a fourth loss corresponding to the second training data based on the atomic displacement information prediction result corresponding to the second training data and the atomic displacement information of the second training data; and determining the second target loss of the catalyst system relaxed energy initial prediction model based on the third loss and the fourth loss corresponding to each piece of second training data in the second training data set.

In some embodiments, the fourth loss may be obtained by calculating a difference in each of dimensions between matrices and then combining differences into a scalar. In some embodiments, the third loss and the fourth loss of each piece of second training data in the second training data set are summed up to obtain a comprehensive loss, then comprehensive losses corresponding to the plurality of pieces of second training data in the second training data set are summed up, and a summation result is determined as the second target loss.

Operation S640: Iteratively update a parameter of the catalyst system relaxed energy initial prediction model based on the second target loss until the second target loss meets a second preset condition, to obtain the catalyst system relaxed energy prediction model.

In some embodiments, the catalyst system relaxed energy initial prediction model may include the graph neural network. Training the catalyst system relaxed energy initial prediction model by using the second training data set includes further training the pre-trained parameter of the graph neural network by using the second training data set, to obtain the graph neural network that can predict both the relaxed energy and the atomic displacement of the catalyst system based on the structure of the catalyst system.

In some embodiments, the second preset condition includes at least one of the following: A current second target loss is less than a predetermined threshold; and a quantity of iterations corresponding to the current second target loss reaches a predetermined quantity. A quantity of times of iteratively updating the parameter of the catalyst system relaxed energy initial prediction model may be less than a quantity of times of iteratively updating the parameter of the catalyst system energy prediction model.

In some embodiments, the catalyst system relaxed energy initial prediction model may include the graph neural network. Training the catalyst system relaxed energy initial prediction model by using the second training data set includes further training the pre-trained parameter of the graph neural network by using the second training data set, to obtain the graph neural network that can predict the relaxed energy of the catalyst system based on the structure of the catalyst system.

In some embodiments, the first output result includes the catalyst system energy prediction result and the atomic force information prediction result; and the catalyst system energy prediction model includes a topology diagram determining sub-model and a graph neural network sub-model, and operation S410 includes the following operations: first, inputting, for each piece of first training data in the first training data set, the first catalyst system structure information corresponding to the first training data to the topology diagram determining sub-model, to obtain a topology diagram corresponding to the first catalyst system structure information, the topology diagram being configured for representing an topological association between atoms in a catalyst system corresponding to the first catalyst system structure information corresponding to the first training data; and then, inputting, for each piece of first training data in the first training data set, the topology diagram corresponding to the first catalyst system structure information corresponding to the first training data to the graph neural network sub-model, to obtain the catalyst system energy prediction result and the atomic force information prediction result. In some embodiments, the graph neural network sub-model includes the graph neural network, and the parameter of the graph neural network is obtained through training.

In some embodiments, the topology diagram includes a plurality of nodes and an edge connecting every two of the plurality of nodes, each node represents an atom in the corresponding catalyst system, and each edge represents an association between two atoms corresponding to the edge. The operation of inputting, for each piece of first training data in the first training data set, the first catalyst system structure information corresponding to the first training data to the topology diagram determining sub-model, to obtain a topology diagram corresponding to the first catalyst system structure information includes the following operations: First, for each piece of first training data in the first training data set, three-dimensional coordinates of the atoms in the catalyst system corresponding to the first catalyst system structure information corresponding to the first training data are determined based on the first catalyst system structure information corresponding to the first training data. In some embodiments, the atoms may include adsorbate atoms, catalyst atoms, and the like. Then, for each piece of first training data in the first training data set, positions of the nodes and a distance between every two of the atoms in the topology diagram corresponding to the first catalyst system structure information are determined based on the three-dimensional coordinates of the atoms in the catalyst system corresponding to the first catalyst system structure information corresponding to the first training data. For each dual-node combination in the topology diagram, in response to that a distance between atoms respectively corresponding to the two nodes in the combination is less than or equal to a predetermined distance threshold, a value of an edge connecting two nodes is 1. For each dual-node combination in the topology diagram, in response to that the distance between the atoms respectively corresponding to the two nodes in the combination is greater than the predetermined distance threshold, the value of the edge connecting the two nodes is 0.

In some embodiments, the obtaining a first training data set includes the following operations: obtaining three-dimensional structure information of each sample catalyst system in a plurality of sample catalyst systems, the three-dimensional structure information including three-dimensional coordinates of atoms in the corresponding sample catalyst system; determining, based on the three-dimensional structure information of each sample catalyst system in the plurality of sample catalyst systems, system energy information and atomic force information of each sample catalyst system by using a quantum mechanical method; and constructing first training data based on the three-dimensional structure information of each sample catalyst system and the system energy information and the atomic force information corresponding to each sample catalyst system. In some embodiments, according to the quantum mechanical method, system energy information and atomic force information of a catalyst system may be calculated through quantum mechanics based on distances between atoms in a structure of the catalyst system.

FIG. 7 is a schematic diagram of training a catalyst system relaxed energy prediction model according to some embodiments of this application. As shown in FIG. 7, first, a 3D structure (including coordinates and sequence numbers of atoms) of a catalyst system and relaxed energy corresponding to the 3D structure of the catalyst system are obtained. For example, the relaxed energy corresponding to the 3D structure of the catalyst system is obtained by calculating a difference between system energy of an initial 3D structure of the catalyst system and system energy of a final 3D structure of the catalyst system. Then, a topology diagram of the catalyst system is constructed based on distances between the atoms. Then, a parameter of a graph neural network is trained by using the topology diagram of the structure of the catalyst system as an input and using the relaxed energy corresponding to the structure of the catalyst system as an expected output (for example, the parameter is fine-tuned based on an initial parameter), to obtain the catalyst system relaxed energy prediction model. In some embodiments, the initial parameter of the graph neural network may be the parameter of the catalyst system energy prediction model.

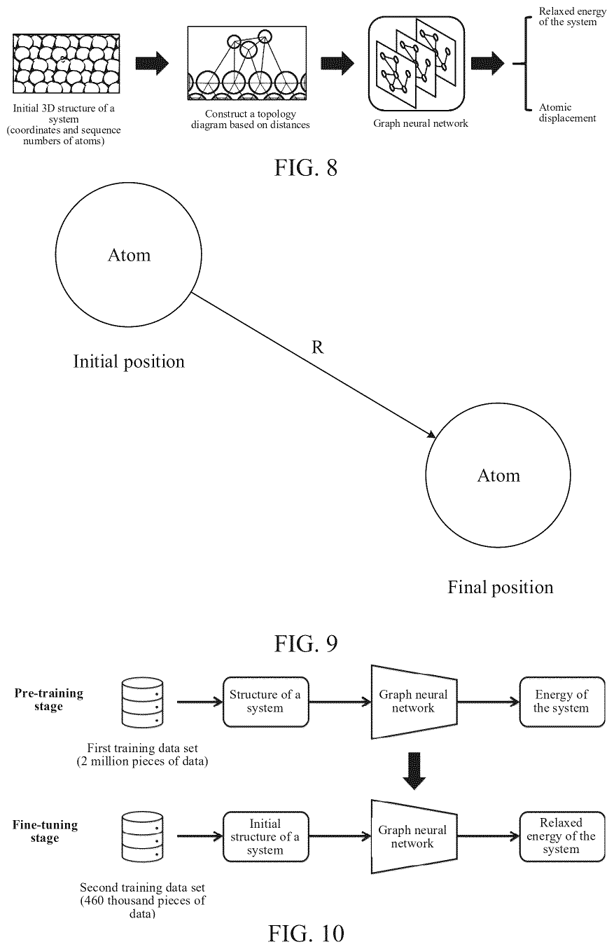

FIG. 8 is a schematic diagram of training a multi-task catalyst system relaxed energy prediction model according to some embodiments of this application. As shown in FIG. 8, first, a 3D structure (including coordinates and sequence numbers of atoms) of a catalyst system, and relaxed energy and atomic displacement corresponding to the 3D structure of the catalyst system are obtained. For example, the relaxed energy corresponding to the 3D structure of the catalyst system is obtained by calculating a difference between system energy of an initial 3D structure of the catalyst system and system energy of a final 3D structure of the catalyst system. The atomic displacement corresponding to the 3D structure of the catalyst system is obtained by calculating differences between coordinates of each of atoms in the initial 3D structure of the catalyst system and coordinates of each of atoms in the final 3D structure of the catalyst system. FIG. 9 is a schematic diagram of atomic displacement according to some embodiments of this application. As shown in FIG. 9, atomic displacement of an atom in the catalyst system may be obtained by subtracting an initial position of the atom from a final position of the atom (that is, a position of the atom when the system is in a relaxed state) (in other words, a vector R in FIG. 9 is obtained).

In some embodiments, atomic displacement information of the catalyst system may be represented in a form of a three-dimensional matrix, and three-dimensional data separately represents mapping of positions of an atom in three directions, that is, a direction x, a direction y, and a direction z. In addition, atomic displacement of the catalyst system may also be represented in a form of a three-dimensional matrix, and three-dimensional data separately represents position changes of an atom in three directions, that is, the direction x, the direction y, and the direction z. It can be found with reference to the foregoing embodiments related to pre-training that, dimensions of atomic displacement are the same as dimensions of an atomic force, and dimensions of relaxed energy are the same as dimensions of system energy. This facilitates quickly obtaining an appropriate parameter through setting during training. In some embodiments, the initial parameter of the graph neural network may be the parameter of the catalyst system energy prediction model (that is, the parameter obtained through pre-training in the foregoing embodiments).

In some embodiments, operation S320 includes the following operation: training, based on a preset first hyperparameter set, the catalyst system energy prediction model by using the first training data set, to obtain the pre-trained catalyst system energy prediction model, the preset first hyperparameter set including a preset first learning rate; and operation S350 includes the following operation: training, based on a preset second hyperparameter set, the catalyst system relaxed energy initial prediction model by using the second training data set, to obtain the catalyst system relaxed energy prediction model, the second hyperparameter set including a preset second learning rate, and the second learning rate being less than the first learning rate. In some embodiments, the second learning rate is equal to or less than one tenth of the first learning rate. Therefore, the learning rate when the catalyst system energy prediction model is trained by using the first training data set is much greater than the learning rate when the catalyst system relaxed energy initial prediction model is trained by using the second training data set because an initial parameter of the latter has been determined by using the former. In this way, the initial parameter only needs to be fine-tuned. It can be learned that, according to the model construction method in the embodiments, the catalyst system relaxed energy prediction model can be constructed more quickly and precisely.

In some embodiments, the first sample label further includes the atomic force information corresponding to the first catalyst system structure information, and the second sample label further includes the atomic displacement information corresponding to the second catalyst system structure information. At a pre-training stage and a fine-tuning stage, trained models are both multi-task prediction models, and output dimensions of the trained models correspond to each other (data dimensions of system energy and relaxed energy are the same, and data dimensions of an atomic force and atomic displacement are the same). Therefore, an initial parameter is determined through pre-training, and then the initial parameter is fine-tuned at the fine-tuning stage, so that an appropriate model parameter can be quickly determined.

This application further discloses a method for predicting relaxed energy of a catalyst system, including: obtaining system structure information of a target catalyst system; and inputting the system structure information of the target catalyst system to the catalyst system relaxed energy prediction model disclosed in any one of the embodiments of this specification, to obtain relaxed energy information of the target catalyst system. In some embodiments, the catalyst system relaxed energy prediction model may be run on the computing device 110 shown in FIG. 1.

FIG. 10 is a schematic diagram of a method for constructing a catalyst system relaxed energy prediction model according to some embodiments of this application, where multi-task training is not performed on the catalyst system relaxed energy prediction model. As shown in FIG. 10, a pre-training stage is first completed by using a first training data set. At the pre-training stage, the first training data set is first obtained. The data set includes 2 million pieces of data, and each piece of data includes a structure of a catalyst system and corresponding system energy. Then, a corresponding topology diagram is determined based on the structure of the catalyst system. A parameter of a graph neural network is trained by using the topology diagram of the structure of the catalyst system as an input and using the system energy corresponding to the structure of the catalyst system as an expected output, to obtain a first parameter of the graph neural network as a pre-training result.

Then, a fine-tuning stage is completed by using a second training data set. The second training data set includes 460 thousand pieces of data, and each piece of data includes an initial structure of the catalyst system and corresponding relaxed energy. Then, a corresponding topology diagram is determined based on the initial structure of the catalyst system. The graph neural network is trained by using the topology diagram of the initial structure of the catalyst system as an input and using the relaxed energy corresponding to the initial structure of the catalyst system as an expected output. A training process is as follows: The parameter of the graph neural network is adjusted from the first parameter until an actual output of the graph neural network approaches the expected output, and the parameter of the graph neural network in this case is determined as a second parameter and is used as a training result. Finally, construction of the catalyst system relaxed energy prediction model is completed. The catalyst system relaxed energy prediction model includes a topology diagram generator and a graph neural network. The topology diagram generator generates a corresponding topology diagram based on a 3D structure of a catalyst system. The graph neural network predicts relaxed energy of the catalyst system based on the topology diagram of the catalyst system. A parameter of the graph neural network is a second parameter.

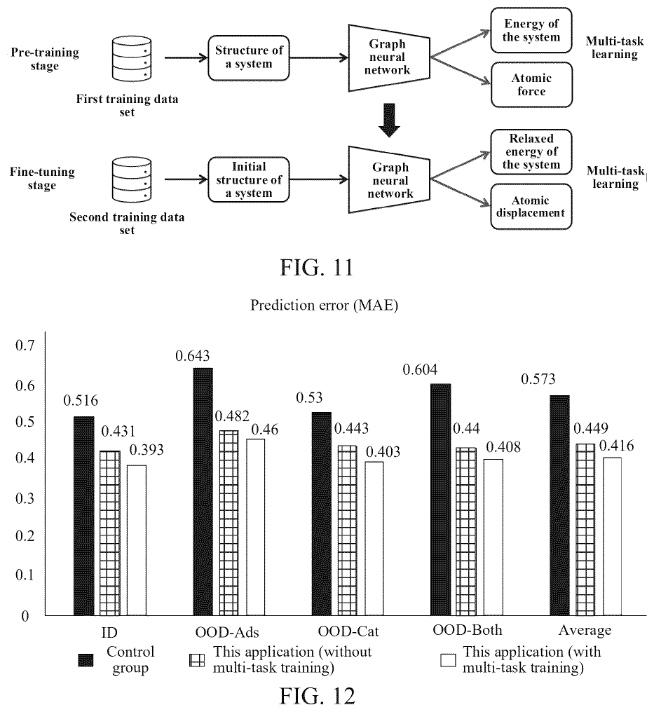

FIG. 11 is a schematic diagram of a method for constructing a multi-task catalyst system relaxed energy prediction model according to some embodiments of this application. First, a pre-training stage is first completed by using a first training data set. At the pre-training stage, the first training data set is first obtained. The data set includes 2 million pieces of data, and each piece of data includes a structure of a catalyst system and corresponding system energy and atomic force. Then, a corresponding topology diagram is determined based on the structure of the catalyst system. A parameter of a graph neural network is trained by using the topology diagram of the structure of the catalyst system as an input and using the system energy and the atomic force corresponding to the structure of the catalyst system as an expected output, to obtain a first parameter of the graph neural network as a pre-training result. It can be learned that, at the pre-training stage, although the input of the graph neural network is only the topology diagram representing the structure of the system, the output of the graph neural network is the system energy and the atomic force. In other words, the graph neural network is trained to perform learning of two tasks. Therefore, the learning belongs to multi-task learning. Multi-task learning facilitates establishment of a closer and more perfect supervision function, so that a process of training the graph neural network is more efficient and quicker, and an obtained parameter is more adaptive.

Then, a fine-tuning stage is completed by using a second training data set. The second training data set includes 460 thousand pieces of data, and each piece of data includes an initial structure of the catalyst system and corresponding relaxed energy and atomic displacement. Then, a corresponding topology diagram is determined based on the initial structure of the catalyst system. The graph neural network is trained by using the topology diagram of the initial structure of the catalyst system as an input and using the relaxed energy and the atomic displacement corresponding to the initial structure of the catalyst system as an expected output. A training process is as follows: The parameter of the graph neural network is adjusted from the first parameter until an actual output of the graph neural network approaches the expected output, and the parameter of the graph neural network in this case is determined as a second parameter and is used as a training result. Finally, construction of the catalyst system relaxed energy prediction model is completed. The catalyst system relaxed energy prediction model includes a topology diagram generator and a graph neural network. The topology diagram generator generates a corresponding topology diagram based on a 3D structure of a catalyst system. The graph neural network predicts relaxed energy and atomic displacement of the catalyst system based on the topology diagram of the catalyst system. A parameter of the graph neural network is a second parameter. It can be learned that, at the fine-tuning stage, although the input of the graph neural network is only the topology diagram representing the structure of the system, the output of the graph neural network is the relaxed energy and the atomic displacement. In other words, the graph neural network is trained to perform learning of two tasks. Therefore, the learning still belongs to multi-task learning. In addition, learning of the two tasks at the fine-tuning stage corresponds to learning of the two tasks at the pre-training stage because outputted data dimensions of the tasks are the same. In this way, the parameter of the graph neural network obtained at the pre-training stage can be used as an initial parameter of the graph neural network at the fine-tuning stage, to quickly determine a most appropriate parameter of the graph neural network at the fine-tuning stage. In this way, the graph neural network obtained through fine-tuning can have higher precision when predicting system relaxed energy and atomic displacement.

TABLE I
Examples of hyperparameters of the graph
neural network at the pre-training stage

| | |
|---|---|
| Layer number | 16 |
| Learning rate | 0.0004 |
| Batch size | 384 |
| Epochs | 20 |

Table I shows hyperparameters of the graph neural network trained at the pre-training stage in the embodiments shown in FIG. 10. As shown in Table I, the layer number of the graph neural network is 16, the learning rate is 0.0004, the batch size is 384, and the epochs are 20. The hyperparameters define a pre-training process. One “epoch” represents a process of training all samples in a training data set once. In one epoch, a training algorithm inputs all the samples to a model according to a set sequence, to perform forward propagation, loss calculation, back propagation, and parameter updating. A batch represents a group of samples inputted to the model at a time. In a process of training a neural network, there are usually many pieces of training data, for example, tens of thousands of pieces of training data or even hundreds of thousands of pieces of training data. If all the tens of thousands of pieces of data are put into a model at a time, requirements on computer performance, a learning capability of a neural network model, and the like are excessively high. Therefore, the training data may be divided into a plurality of batches, and then, samples in each of the batches are inputted to the model together in batches for forward propagation, loss calculation, back propagation, and parameter updating. The “batch size” indicates a quantity of samples in each batch. The learning rate is also referred to as a learning speed, and represents a speed at which information accumulates in the neural network over time. The learning rate is one of hyperparameters that most affect performance. If only one hyperparameter can be adjusted, the best choice is the learning rate. Compared with other hyperparameters, the learning rate controls an effective capacity of the model in a more complex manner. When the learning rate is optimal, the effective capacity of the model is the largest. Therefore, to train the neural network, a key hyperparameter that needs to be set is the learning rate.

TABLE II
Examples of hyperparameters of the graph
neural network at the fine-tuning stage

| | |
|---|---|
| Layer number | 16 |
| Learning rate | 0.00004 |
| Batch size | 384 |
| Epochs | 20 |
| Relaxed energy loss factor | 1 |
| Atomic displacement loss factor | 5 |

Table II shows hyperparameters of the graph neural network trained at the fine-tuning stage in the embodiments shown in FIG. 11. As shown in Table II, the layer number of the graph neural network is 16, the learning rate is 0.00004, the batch size is 384, the epochs are 20, and the relaxed energy loss factor is 1, and the atomic displacement loss factor (delta position loss factor) is 5. The hyperparameters define a training process. It can be found through comparison between Table II and Table I that, the learning rate for training the graph neural network at the fine-tuning stage is one tenth of that at the pre-training stage. This is because the initial parameter of the graph neural network at the fine-tuning stage is the training result at the pre-training stage and is already not far away from a target result, and therefore only needs to be fine-tuned. Therefore, the learning rate at the fine-tuning stage may be set to be much smaller than that at the pre-training stage. It can be learned that, at the fine-tuning stage, an output head that is in an “atom-force” model and that is configured for predicting energy of a system is used to predict relaxed energy of the system, and an output head that is in the “atom-force” model and that is configured for predicting an atomic force of the system is also used to predict atomic displacement of the system. Predicting the relaxed energy of the system is a graph-level task, and predicting the atomic displacement of the system is a node-level task. The two may be simultaneously trained. Such a process is multi-task training. Predicting a position offset of the system is actually an auxiliary task, and addition of the task can further improve precision of predicting the relaxed energy of the system by the model. It can be learned that, when a model is constructed according to the operations in the embodiments, an initial parameter of the model may be quickly determined first, and then the initial parameter is fine-tuned, to efficiently and precisely construct the model.

TABLE III
Hyperparameters of the graph neural network
trained at the pre-training stage

| | |
|---|---|
| Layer number | 16 |
| Learning rate | 0.0004 |
| Batch size | 384 |
| Epochs | 20 |

Table III shows the hyperparameters of the graph neural network trained at the pre-training stage in the embodiments shown in FIG. 8. As shown in Table III, the layer number of the graph neural network is 16, the learning rate is 0.0004, the batch size is 384, and the epochs are 20. The hyperparameters define a pre-training process. As mentioned above, in a process of training a neural network, training data usually needs to be divided into a plurality of batches. A quantity of samples specifically included in each of the batches is specified by the batch size. The learning rate is also referred to as a learning speed, and represents a speed at which information accumulates in the neural network over time. The learning rate is one of hyperparameters that most affect performance. If only one hyperparameter can be adjusted, the best choice is the learning rate. Compared with other hyperparameters, the learning rate controls an effective capacity of a model in a more complex manner. When the learning rate is optimal, the effective capacity of the model is the largest. Therefore, to train the neural network, a key hyperparameter that needs to be set is the learning rate.

TABLE IV
Hyperparameters of the graph neural network
trained at the fine-tuning stage

| | |
|---|---|
| Layer number | 16 |
| Learning rate | 0.00004 |
| Batch size | 384 |
| Epochs | 20 |

Table IV shows the hyperparameters the graph neural network trained at the fine-tuning stage in the embodiments shown in FIG. 7. As shown in Table IV, the layer number of the graph neural network is 16, the learning rate is 0.00004, the batch size is 384, and the epochs are 20. The hyperparameters define a training process. It can be found through comparison between Table IV and Table III that, the learning rate for training the graph neural network at the fine-tuning stage is one tenth of that at the pre-training stage. This is because the initial parameter of the graph neural network at the fine-tuning stage is the training result at the pre-training stage and is not far away from a target result. In this way, only fine-tuning needs to be performed. Therefore, the learning rate at the fine-tuning stage may be set to be much smaller than that at the pre-training stage. It can be learned that, when a model is constructed according to the operations in the embodiments, an initial parameter of the model may be quickly determined first, and then fine-tuning is performed based on the initial parameter, to efficiently and precisely construct the model.

FIG. 12 is a diagram of an effect of a catalyst system relaxed energy prediction model according to some embodiments of this application. In FIG. 12, a control group selects a catalyst system relaxed energy prediction model directly constructed without pre-training. In the embodiments of this application, a catalyst system relaxed energy prediction model constructed through pre-training without multi-task training shown in FIG. 10 and a catalyst system relaxed energy prediction model constructed through multi-task training shown in FIG. 11 are used. Four authoritative test sets in the industry are selected, which are an in domain (ID) test set, an out of domain adsorbate (OOD-Ads) test set, an out of domain catalyst (OOD-Cat) test set, and an out of domain both (OOD-Both) test set. Data volumes of the four authoritative test sets are shown in Table V.

TABLE V
Examples of the authoritative test sets

| Test set | Data volume |
|---|---|
| ID | 25000 |
| OOD-Ads | 25000 |
| OOD-Cat | 25000 |
| OOD-Both | 25000 |

The four authoritative test sets shown in Table V are used to perform tests on the control group and the catalyst relaxed energy prediction models constructed in the embodiments of this application, and a test result is shown in FIG. 12. Prediction precision is measured by using a prediction error, and the prediction error is measured by using a mean absolute error (MAE) indicator. A lower value of the indicator indicates higher prediction precision of a model. In FIG. 12, a black bar represents the control group, a grid-shaped bar represents an embodiment of this application without multi-task training (referred to as a solution 1 of this application), and a white bar represents an embodiment of this application with multi-task training (referred to as a solution 2 of this application). It can be apparently learned that, in the four test sets, both prediction precision of the solution 1 of this application and prediction precision of the solution 2 of this application are higher than that of the control group. In addition, the figure shows averages of four groups. It can be learned from the averages that, compared with the control group, average precision of prediction precision of the model in the solution 1 of this application is improved by 21.6%; compared with the control group, average precision of prediction precision of the model in the solution 2 of this application is improved by 27.4%; and compared with the solution 1 of this application, the average precision of the prediction precision of the model in the solution 2 of this application is improved by 7.3%. It can be learned that, precision of a model obtained by using the method for constructing a catalyst system relaxed energy prediction model disclosed in this application is higher than that of a model obtained by using a related technical solution, and precision of a catalyst system relaxed energy prediction model obtained through multi-task training is higher.

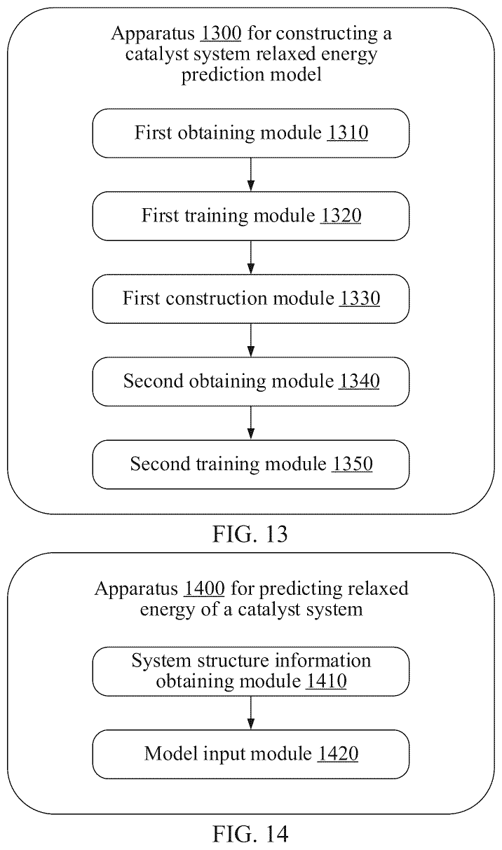

FIG. 13 is an exemplary structural block diagram of an apparatus 1300 for constructing a catalyst system relaxed energy prediction model according to some embodiments of this application.

As shown in FIG. 13, the apparatus 1300 for constructing a catalyst system relaxed energy prediction model includes a first obtaining module 1310, a first training module 1320, a first construction module 1330, a second obtaining module 1340, and a second training module 1350.

The first obtaining module 1310 is configured to obtain a first training data set, the first training data set including a plurality of pieces of first training data, each piece of first training data including a first training sample and a corresponding first sample label, the first training sample including first catalyst system structure information, and the first sample label including system energy information corresponding to the first catalyst system structure information.

The first training module 1320 is configured to train a catalyst system energy prediction model by using the first training data set, to obtain a pre-trained catalyst system energy prediction model.

The first construction module 1330 is configured to train a catalyst system energy prediction model by using the first training data set, to obtain a pre-trained catalyst system energy prediction model.

The second obtaining module 1340 is configured to obtain a second training data set, the second training data set including a plurality of pieces of second training data, each piece of second training data including a second training sample and a corresponding second sample label, the second training sample including second catalyst system structure information, and the second sample label including relaxed energy information corresponding to the second catalyst system structure information.

The second training module 1350 is configured to train the catalyst system relaxed energy initial prediction model by using the second training data set, to obtain a catalyst system relaxed energy prediction model.

Therefore, in the apparatus 1300 for constructing a catalyst system relaxed energy prediction model, a catalyst system relaxed energy initial prediction model is constructed based on a pre-trained catalyst system energy prediction model, so that the catalyst system relaxed energy initial prediction model can be quickly determined. Then, a second training module 1350 adjusts the catalyst system relaxed energy initial prediction model by using a second training data set, to obtain a catalyst system relaxed energy prediction model, so that the catalyst system relaxed energy prediction model with high precision and a fast response can be quickly obtained. Therefore, according to the apparatus 1300 for constructing a catalyst system relaxed energy prediction model, a high-quality catalyst system relaxed energy prediction model can be constructed quickly and easily.

In some embodiments, the first sample label further includes atomic force information corresponding to the first catalyst system structure information, and the second sample label further includes atomic displacement information corresponding to the second catalyst system structure information.

In some embodiments, the first training module 1320 is further configured to input, for each piece of first training data in the first training data set, the first training sample of the first training data to the catalyst system energy prediction model, to obtain a first output result corresponding to the first training data; calculate, for each piece of first training data in the first training data set, a first loss corresponding to the first training data based on the first output result corresponding to the first training data and the first sample label of the first training data; determine a first target loss of the catalyst system energy prediction model based on the first loss corresponding to each piece of first training data in the first training data set; and iteratively update a parameter of the catalyst system energy prediction model based on the first target loss until the first target loss meets a first preset condition, to obtain the pre-trained catalyst system energy prediction model.

In some embodiments, when the first sample label further includes the atomic force information corresponding to the first catalyst system structure information, the first output result includes a catalyst system energy prediction result and an atomic force information prediction result; and the first training module 1320 is further configured to calculate, for each piece of first training data in the first training data set, a first sub-loss based on the catalyst system energy prediction result corresponding to the first training data and the system energy information of the first training data; calculate, for each piece of first training data in the first training data set, a second sub-loss corresponding to the first training data based on the atomic force information prediction result corresponding to the first training data and the atomic force information of the first training data; and determine, for each piece of first training data in the first training data set, the first loss based on the first sub-loss and the second sub-loss corresponding to the first training data.

In some embodiments, when the first sample label further includes the atomic force information corresponding to the first catalyst system structure information, the first output result includes the catalyst system energy prediction result and the atomic force information prediction result; and the first training module 1320 is further configured to calculate, for each piece of first training data in the first training data set, a second loss corresponding to the first training data based on the atomic force information prediction result corresponding to the first training data and the atomic force information of the first training data; and determine the first target loss of the catalyst system energy prediction model based on the first loss and the second loss corresponding to each piece of first training data in the first training data set.

In some embodiments, the first output result includes the catalyst system energy prediction result and the atomic force information prediction result; and the catalyst system energy prediction model includes a topology diagram determining sub-model and a graph neural network sub-model, and the first training module 1320 is further configured to input, for each piece of first training data in the first training data set, the first catalyst system structure information corresponding to the first training data to the topology diagram determining sub-model, to obtain a topology diagram corresponding to the first catalyst system structure information, the topology diagram being configured for representing an topological association between atoms in a catalyst system corresponding to the first catalyst system structure information corresponding to the first training data; and input, for each piece of first training data in the first training data set, the topology diagram corresponding to the first catalyst system structure information corresponding to the first training data to the graph neural network sub-model, to obtain the catalyst system energy prediction result and the atomic force information prediction result.

In some embodiments, the topology diagram includes a plurality of nodes and an edge connecting every two of the plurality of nodes, each node represents an atom in the corresponding catalyst system, and each edge represents an association between two atoms corresponding to the edge; and the first training module 1320 is further configured to determine, for each piece of first training data in the first training data set, three-dimensional coordinates of the atoms in the catalyst system corresponding to the first catalyst system structure information corresponding to the first training data based on the first catalyst system structure information corresponding to the first training data; and determine, for each piece of first training data in the first training data set, positions of the nodes and a distance between every two of the atoms in the topology diagram corresponding to the first catalyst system structure information based on the three-dimensional coordinates of the atoms in the catalyst system corresponding to the first catalyst system structure information corresponding to the first training data, for each dual-node combination in the topology diagram, in response to that a distance between atoms respectively corresponding to the two nodes in the combination is less than or equal to a predetermined distance threshold, a value of an edge connecting two nodes being 1; or for each dual-node combination in the topology diagram, in response to that the distance between the atoms respectively corresponding to the two nodes in the combination is greater than the predetermined distance threshold, the value of the edge connecting the two nodes being 0.

In some embodiments, the second training module 1350 is further configured to input, for each piece of second training data in the second training data set, the second catalyst system structure information of the second training data to the catalyst system relaxed energy initial prediction model, to obtain a second output result corresponding to the second training data; calculate, for each piece of second training data in the second training data set, a third loss corresponding to the second training data based on the second output result corresponding to the second training data and the second sample label; determine a second target loss of the catalyst system relaxed energy initial prediction model based on the third loss corresponding to each piece of second training data in the second training data set; and iteratively update a parameter of the catalyst system relaxed energy initial prediction model based on the second target loss until the second target loss meets a second preset condition, to obtain the catalyst system relaxed energy prediction model.

In some embodiments, the second output result includes a catalyst system relaxed energy prediction result; and the second training module 1350 is further configured to calculate, for each piece of second training data in the second training data set, the third loss corresponding to the second training data based on the catalyst system relaxed energy prediction result corresponding to the second training data and the relaxed energy information of the second training data.

In some embodiments, the second sample label further includes the atomic displacement information corresponding to the second catalyst system structure information; the second output result further includes an atomic displacement information prediction result; and the second training module 1350 is further configured to calculate, for each piece of second training data in the second training data set, a fourth loss corresponding to the second training data based on the atomic displacement information prediction result corresponding to the second training data and the atomic displacement information of the second training data; and determine the second target loss of the catalyst system relaxed energy initial prediction model based on the third loss and the fourth loss corresponding to each piece of second training data in the second training data set.

In some embodiments, the first obtaining module 1310 is further configured to obtain three-dimensional structure information of each sample catalyst system in a plurality of sample catalyst systems, the three-dimensional structure information including three-dimensional coordinates of atoms in the corresponding sample catalyst system; determine, based on the three-dimensional structure information of each sample catalyst system in the plurality of sample catalyst systems, system energy information and atomic force information of each sample catalyst system by using a quantum mechanical method; and construct first training data based on the three-dimensional structure information of each sample catalyst system and the system energy information and the atomic force information corresponding to each sample catalyst system.

In some embodiments, the first training module 1320 is further configured to train, based on a preset first hyperparameter set, the catalyst system energy prediction model by using the first training data set, to obtain the pre-trained catalyst system energy prediction model, the preset first hyperparameter set including a preset first learning rate.

In some embodiments, the first training module 1320 is further configured to train, based on a preset second hyperparameter set, the catalyst system relaxed energy initial prediction model by using the second training data set, to obtain the catalyst system relaxed energy prediction model, the second hyperparameter set including a preset second learning rate, and the second learning rate being less than the first learning rate.

In some embodiments, the second learning rate is equal to or less than one tenth of the first learning rate.

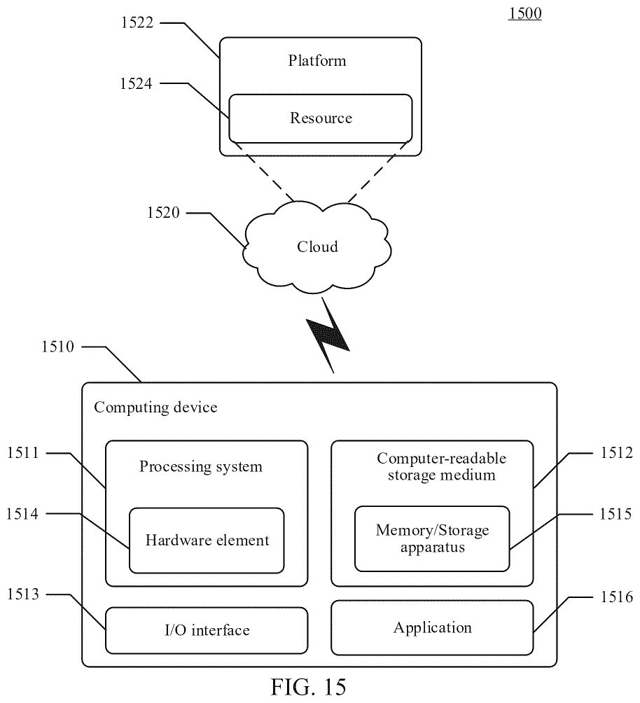

As shown in FIG. 14, this application further provides an apparatus 1400 for predicting relaxed energy of a catalyst system, including a system structure information obtaining module 1410 and a model input module 1420.

The system structure information obtaining module 1410 is configured to obtain system structure information of a target catalyst system.

The model input module 1420 is configured to input the system structure information of the target catalyst system to the catalyst system relaxed energy prediction model obtained by using the method for constructing a catalyst system relaxed energy prediction model in any one of the embodiments, to obtain relaxed energy information of the target catalyst system.

FIG. 15 shows an exemplary system 1500 including an exemplary computing device 1510 representing one or more systems and/or devices that can implement various methods described in this specification. The computing device 1510 may be, for example, a server of a service provider, a device associated with the server, an on-chip system, and/or any other appropriate computing device or computing system. The apparatus 1300 for constructing a catalyst system relaxed energy prediction model described above with reference to FIG. 9 may use a form of the computing device 1510. Alternatively, the apparatus 1300 for constructing a catalyst system relaxed energy prediction model may be implemented as a computer program in a form of an application 1516.

The exemplary computing device 1510 shown in the figure includes a processing system 1511, one or more computer-readable media 1512, and one or more I/O interfaces 1513 that are communicatively coupled to each other. Although not shown in the figure, the computing device 1510 may further include a system bus or another data and command transfer system, which couples various components to each other. The system bus may include any one or a combination of different bus structures. The bus structure is, for example, a memory bus or a memory controller, a peripheral bus, a universal serial bus, and/or a processor or a local bus using any one of various bus architectures. Various other examples such as a control line and a data line are also conceived.

The processing system 1511 represents a function of performing one or more operations by using hardware. Therefore, the processing system 1511 is shown to include a hardware element 1514 that may be configured as a processor, a functional block, or the like. The processing system 1511 may include another logical device implemented as an application-specific integrated circuit in hardware or formed by using one or more semiconductors. The hardware element 1514 is not limited by a material by which the hardware element 1514 is formed or a processing mechanism of which the hardware element 1514 uses. For example, the processor may be formed by one or more semiconductors and/or transistors (for example, an electronic integrated circuit (IC)). In such context, processor-executable instructions may be electronic executable instructions.

The computer-readable medium 1512 is shown to include a memory/storage apparatus 1515. The memory/storage apparatus 1515 represents a memory/storage capacity associated with one or more computer-readable media. The memory/storage apparatus 1515 may include a volatile medium (for example, a random access memory (RAM)) and/or a non-volatile medium (for example, a read-only memory (ROM), a flash memory, an optical disc, or a magnetic disk). The memory/storage apparatus 1515 may include a fixed medium (for example, a RAM, a ROM, or a fixed hard disk drive) and a removable medium (for example, a flash memory, a removable hard disk drive, or an optical disc). The computer-readable medium 1512 may be configured in various other manners further described below.

The one or more I/O interfaces 1513 represent functions of allowing a user to input a command and information to the computing device 1510 by using various input devices and also allowing to present information to the user and/or other components or devices by using various output devices. Examples of the input device include a keyboard, a cursor control device (for example, a mouse), a microphone (for example, used for a voice input), a scanner, a touch function (for example, a capacitive sensor or another sensor configured to detect a physical touch), a camera (for example, a motion not related to a touch may be detected as a gesture by using a visible or invisible wave length (for example, an infrared frequency)), and the like. Examples of the output device include a display device, a speaker, a printer, a network adapter, a tactile response device, and the like. Therefore, the computing device 1510 may be configured in various manners further described below to support user interaction.

The computing device 1510 further includes the application 1516. The application 1516 may be, for example, an instance of software used for the apparatus 1300 for constructing a catalyst system relaxed energy prediction model, and implements the technologies described in this specification in combination with other elements in the computing device 1510.

This application provides a computer program product or a computer program. The computer program product or the computer program includes computer instructions, the computer instructions being stored in a non-transitory computer-readable storage medium. A processor of a computing device reads the computer instructions from the computer-readable storage medium, and the processor executes the computer instructions, to cause the computing device to perform the method for constructing a catalyst system relaxed energy prediction model provided in the various exemplary implementations.

The technologies in this specification may be described in general context of software and hardware elements or program modules. Generally, the modules include a routine, a program, an object, an element, a component, a data structure, and the like for executing a particular task or implementing a particular abstract data type. Terms “module”, “function”, and “component” used in this specification generally represent software, firmware, hardware, or a combination thereof. Features of the technologies described in this specification are platform-independent, meaning that the technologies may be implemented on various computing platforms having various processors.

Implementations of the described modules and technologies may be stored on a computer-readable medium in a form or may be transmitted across a computer-readable medium in a form. The computer-readable media may include various media that can be accessed by the computing device 1510. In some embodiments, not for limiting, the computer-readable medium may include a “computer-readable storage medium” and a “computer-readable signal medium”.

In contrast to mere signal transmission, carriers, or signals, the “computer-readable storage medium” is a medium and/or a device that can persistently store information, and/or a tangible storage apparatus. Therefore, a non-transitory computer-readable storage medium is a non-signal-carrying medium. The computer-readable storage medium includes hardware such as volatile and non-volatile media, removable and non-removable media, and/or a storage device implemented by using a method or technology suitable for storing information (such as computer-readable instructions, a data structure, a program module, a logical element/circuit, or other data). Examples of the computer-readable storage medium may include, but are not limited to, a RAM, a ROM, an erasable programmable read only memory (EEPROM), a flash memory or another memory technology, a compact disc read-only memory (CD-ROM), a digital versatile disc (DVD) or another optical storage apparatus, a hard disk, a cassette, a magnetic tape, a magnetic disk storage apparatus or another magnetic storage device, another storage device, a tangible medium, or an article suitable for storing expected information and accessible by a computer.

The “computer-readable signal medium” is a signal-carrying medium configured to send instructions to hardware of the computing device 1510 through, for example, a network. Typically, the signal medium may embody computer-readable instructions, a data structure, a program module, or other data in, for example, a carrier, a data signal, or a modulated data signal of another transmission mechanism. The signal medium further includes any information transfer medium. The term “modulated data signal” is such a signal, where one or more features of the signal are set or changed, to encode information into the signal. In some embodiments, not for limiting, a communication medium includes a wired medium, for example, a wired network or a direct connection, and a wireless medium, for example, sound, a radio frequency (RF), or an infrared ray.

As described above, the hardware element 1514 and the computer-readable medium 1512 represent instructions, modules, programmable device logic, and/or fixed device logic implemented in a hardware form, and in some embodiments, may be used to implement at least some aspects of the technologies described in this specification. The hardware element may include an integrated circuit or an on-chip system, an application-specific integrated circuit (ASIC), a field programmable gate array (FPGA), a complex programmable logic device (CPLD), and another implementation in silicon or a component of another hardware device. In such context, the hardware element may be used as a processing device configured to execute a program task defined by an instruction, a module, and/or logic embodied by the hardware element, and may be used as a hardware device configured to store an executed instruction, for example, the computer-readable storage medium described above.

A combination of the foregoing may also be used to implement various technologies and modules in this specification. Therefore, a software module, a hardware module, or a program module and another program module may be implemented as one or more instructions and/or logic that is on a non-transitory computer-readable storage medium in a form and/or that is embodied by one or more hardware elements 1514. The computing device 1510 may be configured to implement particular instructions and/or functions corresponding to the software module and/or the hardware module. Therefore, for example, by using the computer-readable storage medium and/or the hardware element 1514 of the processing system, modules may be at least partially implemented in hardware as modules that can be executed by the computing device 1510 as software. The instructions and/or functions may be executed/operated by one or more articles (for example, one or more computing devices 1510 and/or one or more processing systems 1511) to implement the technologies, modules, and examples described in this specification.

In various implementations, the computing device 1510 may use various different configurations. For example, the computing device 1510 may be implemented as a computer-type device including a personal computer, a desktop computer, a multi-screen computer, a laptop computer, a netbook, and the like. The computing device 1510 may alternatively be implemented as a mobile apparatus-type device including mobile devices such as a mobile phone, a portable music player, a portable game device, a tablet computer, and a multi-screen computer. The computing device 1510 may alternatively be implemented as a television-type device, including devices having or being connected to a generally large screen in a casual watching environment. The devices include a television, a set-top box, a game console, and the like.

The technologies described in this specification may be supported by these various configurations of the computing device 1510, and are not limited to specific examples of the technologies described in this specification. The functions may alternatively be entirely or partially implemented on a cloud 1520 by using a distributed system, for example, by using a platform 1522 described below.

The cloud 1520 includes and/or represents the platform 1522 for a resource 1524. The platform 1522 abstracts underlying functions of hardware (for example, servers) and software resources of the cloud 1520. The resource 1524 may include an application and/or data that can be used when computer processing is executed on a server far away from the computing device 1510. The resource 1524 may further include services provided through the Internet and/or through a subscriber network like a cellular network or Wi-Fi network.

The platform 1522 may abstract resources and functions to connect the computing device 1510 with other computing devices. The platform 1522 may also be configured to abstract scaling of resources to provide a corresponding level of scale to encountered demand for the resource 1524 that is implemented via the platform 1522. Therefore, in an embodiment of an interconnected device, implementations of functions described in this specification may be distributed in the entire system 1500. For example, the functions may be partially implemented on the computing device 1510 and implemented through the platform 1522 that abstracts the functions of the cloud 1520.

For clarity, the embodiments of this application are described with reference to different functional units. However, apparently, functionality of each functional unit may be implemented in a single unit, implemented in a plurality of units, or implemented as a part of another functional unit without departing from this application. For example, functionality described as being performed by a single unit may be performed by a plurality of different units. Therefore, reference to a particular functional unit is only considered as reference to an appropriate unit configured to provide the described functionality, rather than indicating a strict logical or physical structure or organization. Therefore, this application may be implemented in a single unit, or may be physically and functionally distributed between different units and circuits.

Although this application has been described with reference to some embodiments, this application is not intended to be limited to specific forms described in this specification. On the contrary, the scope of this application is limited by the appended claims only. In addition, although individual features may be included in different claims, these may be combined favorably, and a combination that is included in different claims and that does not imply a feature is not feasible and/or favorable. The order of the features in the claims does not imply any particular order in which the features necessarily work. In addition, in the claims, the term “comprise” does not exclude other elements, and the term “a” or “one” does not exclude a plurality. Reference signs in the claims are provided only as clear examples, and are not to be construed as limiting the scope of the claims in any manner.

Data related to a test case of software and the like is involved in specific implementations of this application. When the foregoing embodiments of this application are applied to a specific product or technology, the user's permission or consent needs to be obtained, and collection, use, and processing of the relevant data need to comply with relevant laws, regulations, and standards of relevant countries and regions.

Technical features of the foregoing embodiments may be combined in different manners to form other embodiments. To make description concise, not all possible combinations of the technical features in the foregoing embodiments are described. However, the combinations of these technical features shall be considered as falling within the scope recorded by this specification provided that no conflict exists.

The foregoing embodiments only describe several implementations of this application, which are described specifically and in detail, but cannot be construed as a limitation to the patent scope of this application. A person of ordinary skill in the art may further make several variations and improvements without departing from the ideas of this application, and such variations and improvements all fall within the protection scope of this application. Therefore, the protection scope of this application is subject to the protection scope of the appended claims.

What is claimed is:

1. A method for constructing a catalyst system relaxed energy prediction model performed by a computing device, the method comprising:
obtaining a first training data set, the first training data set comprising a plurality of pieces of first training data, each piece of first training data comprising a first training sample and a corresponding first sample label;
training a catalyst system energy prediction model by using the first training data set, to obtain a pre-trained catalyst system energy prediction model;
constructing a catalyst system relaxed energy initial prediction model based on the pre-trained catalyst system energy prediction model;
obtaining a second training data set, the second training data set comprising a plurality of pieces of second training data, each piece of second training data comprising a second training sample and a corresponding second sample label; and
training the catalyst system relaxed energy initial prediction model by using the second training data set, to obtain a catalyst system relaxed energy prediction model.

2. The method according to claim 1, wherein the first sample label further comprises atomic force information corresponding to the first training sample, and the second sample label further comprises atomic displacement information corresponding to the second training sample.

3. The method according to claim 1, wherein the training a catalyst system energy prediction model by using the first training data set, to obtain a pre-trained catalyst system energy prediction model comprises:
inputting, for each piece of first training data in the first training data set, the first training sample of the first training data to the catalyst system energy prediction model, to obtain a first output result corresponding to the first training data;
calculating, for each piece of first training data in the first training data set, a first loss corresponding to the first training data based on the first output result corresponding to the first training data and the first sample label of the first training data;
determining a first target loss of the catalyst system energy prediction model based on the first loss corresponding to each piece of first training data in the first training data set; and
iteratively updating a parameter of the catalyst system energy prediction model based on the first target loss until the first target loss meets a first preset condition, to obtain the pre-trained catalyst system energy prediction model.

4. The method according to claim 1, wherein the obtaining a first training data set comprises:
obtaining three-dimensional structure information of each sample catalyst system in a plurality of sample catalyst systems, the three-dimensional structure information comprising three-dimensional coordinates of atoms in the corresponding sample catalyst system;
determining, based on the three-dimensional structure information of each sample catalyst system in the plurality of sample catalyst systems, system energy information and atomic force information of each sample catalyst system by using a quantum mechanical method; and
constructing first training data based on the three-dimensional structure information of each sample catalyst system and the system energy information and the atomic force information corresponding to each sample catalyst system.

5. The method according to claim 1, wherein the training a catalyst system energy prediction model by using the first training data set, to obtain a pre-trained catalyst system energy prediction model comprises:
training, based on a preset first hyperparameter set, the catalyst system energy prediction model by using the first training data set, to obtain the pre-trained catalyst system energy prediction model, the preset first hyperparameter set comprising a preset first learning rate.

6. The method according to claim 1, wherein the training the catalyst system relaxed energy initial prediction model by using the second training data set, to obtain a catalyst system relaxed energy prediction model comprises:
training, based on a preset second hyperparameter set, the catalyst system relaxed energy initial prediction model by using the second training data set, to obtain the catalyst system relaxed energy prediction model, the second hyperparameter set comprising a preset second learning rate, and the second learning rate being less than the first learning rate.

7. The method according to claim 1, wherein the method further comprises:
obtaining system structure information of a target catalyst system; and
inputting the system structure information of the target catalyst system to the catalyst system relaxed energy prediction model to obtain relaxed energy information of the target catalyst system.

8. A computing device, comprising:
a memory, configured to store computer-executable instructions; and
a processor, configured to perform, when the computer-executable instructions are executed by the processor, a method for constructing a catalyst system relaxed energy prediction model including:
obtaining a first training data set, the first training data set comprising a plurality of pieces of first training data, each piece of first training data comprising a first training sample and a corresponding first sample label;
training a catalyst system energy prediction model by using the first training data set, to obtain a pre-trained catalyst system energy prediction model;
constructing a catalyst system relaxed energy initial prediction model based on the pre-trained catalyst system energy prediction model;
obtaining a second training data set, the second training data set comprising a plurality of pieces of second training data, each piece of second training data comprising a second training sample and a corresponding second sample label; and
training the catalyst system relaxed energy initial prediction model by using the second training data set, to obtain a catalyst system relaxed energy prediction model.

9. The computing device according to claim 8, wherein the first sample label further comprises atomic force information corresponding to the first training sample, and the second sample label further comprises atomic displacement information corresponding to the second training sample.

10. The computing device according to claim 8, wherein the training a catalyst system energy prediction model by using the first training data set, to obtain a pre-trained catalyst system energy prediction model comprises:
inputting, for each piece of first training data in the first training data set, the first training sample of the first training data to the catalyst system energy prediction model, to obtain a first output result corresponding to the first training data;
calculating, for each piece of first training data in the first training data set, a first loss corresponding to the first training data based on the first output result corresponding to the first training data and the first sample label of the first training data;
determining a first target loss of the catalyst system energy prediction model based on the first loss corresponding to each piece of first training data in the first training data set; and
iteratively updating a parameter of the catalyst system energy prediction model based on the first target loss until the first target loss meets a first preset condition, to obtain the pre-trained catalyst system energy prediction model.

11. The computing device according to claim 8, wherein the obtaining a first training data set comprises:
obtaining three-dimensional structure information of each sample catalyst system in a plurality of sample catalyst systems, the three-dimensional structure information comprising three-dimensional coordinates of atoms in the corresponding sample catalyst system;
determining, based on the three-dimensional structure information of each sample catalyst system in the plurality of sample catalyst systems, system energy information and atomic force information of each sample catalyst system by using a quantum mechanical method; and
constructing first training data based on the three-dimensional structure information of each sample catalyst system and the system energy information and the atomic force information corresponding to each sample catalyst system.

12. The computing device according to claim 8, wherein the training a catalyst system energy prediction model by using the first training data set, to obtain a pre-trained catalyst system energy prediction model comprises:
training, based on a preset first hyperparameter set, the catalyst system energy prediction model by using the first training data set, to obtain the pre-trained catalyst system energy prediction model, the preset first hyperparameter set comprising a preset first learning rate.

13. The computing device according to claim 8, wherein the training the catalyst system relaxed energy initial prediction model by using the second training data set, to obtain a catalyst system relaxed energy prediction model comprises:
training, based on a preset second hyperparameter set, the catalyst system relaxed energy initial prediction model by using the second training data set, to obtain the catalyst system relaxed energy prediction model, the second hyperparameter set comprising a preset second learning rate, and the second learning rate being less than the first learning rate.

14. The computing device according to claim 8, wherein the method further comprises:
obtaining system structure information of a target catalyst system; and
inputting the system structure information of the target catalyst system to the catalyst system relaxed energy prediction model to obtain relaxed energy information of the target catalyst system.

15. A non-transitory computer-readable storage medium, having computer-executable instructions stored therein, the computer-executable instructions, when executed, implementing a method for constructing a catalyst system relaxed energy prediction model obtaining a first training data set, the first training data set comprising a plurality of pieces of first training data, each piece of first training data comprising a first training sample and a corresponding first sample label;
training a catalyst system energy prediction model by using the first training data set, to obtain a pre-trained catalyst system energy prediction model;
constructing a catalyst system relaxed energy initial prediction model based on the pre-trained catalyst system energy prediction model;
obtaining a second training data set, the second training data set comprising a plurality of pieces of second training data, each piece of second training data comprising a second training sample and a corresponding second sample label; and
training the catalyst system relaxed energy initial prediction model by using the second training data set, to obtain a catalyst system relaxed energy prediction model.

16. The non-transitory computer-readable storage medium according to claim 15, wherein the first sample label further comprises atomic force information corresponding to the first training sample, and the second sample label further comprises atomic displacement information corresponding to the second training sample.

17. The non-transitory computer-readable storage medium according to claim 15, wherein the training a catalyst system energy prediction model by using the first training data set, to obtain a pre-trained catalyst system energy prediction model comprises:
inputting, for each piece of first training data in the first training data set, the first training sample of the first training data to the catalyst system energy prediction model, to obtain a first output result corresponding to the first training data;
calculating, for each piece of first training data in the first training data set, a first loss corresponding to the first training data based on the first output result corresponding to the first training data and the first sample label of the first training data;
determining a first target loss of the catalyst system energy prediction model based on the first loss corresponding to each piece of first training data in the first training data set; and
iteratively updating a parameter of the catalyst system energy prediction model based on the first target loss until the first target loss meets a first preset condition, to obtain the pre-trained catalyst system energy prediction model.

18. The non-transitory computer-readable storage medium according to claim 15, wherein the obtaining a first training data set comprises:
obtaining three-dimensional structure information of each sample catalyst system in a plurality of sample catalyst systems, the three-dimensional structure information comprising three-dimensional coordinates of atoms in the corresponding sample catalyst system;
determining, based on the three-dimensional structure information of each sample catalyst system in the plurality of sample catalyst systems, system energy information and atomic force information of each sample catalyst system by using a quantum mechanical method; and
constructing first training data based on the three-dimensional structure information of each sample catalyst system and the system energy information and the atomic force information corresponding to each sample catalyst system.

19. The non-transitory computer-readable storage medium according to claim 15, wherein the training a catalyst system energy prediction model by using the first training data set, to obtain a pre-trained catalyst system energy prediction model comprises:
training, based on a preset first hyperparameter set, the catalyst system energy prediction model by using the first training data set, to obtain the pre-trained catalyst system energy prediction model, the preset first hyperparameter set comprising a preset first learning rate.

20. The non-transitory computer-readable storage medium according to claim 15, wherein the method further comprises:
obtaining system structure information of a target catalyst system; and
inputting the system structure information of the target catalyst system to the catalyst system relaxed energy prediction model to obtain relaxed energy information of the target catalyst system.
